(19) United States
(12) Patent Application Publication
(10) Pub. No.: US 2021/0244710 A1
(43) Pub. Date: Aug. 12, 2021

(54) STABLE PHARMACEUTICAL FORMULATIONS OF OXYMETAZOLINE

(71) Applicants: Kangping XIAO, Westfield, NJ (US); Qiuxia ZHAO, Bernardsville, NJ (US); BAYER HEALTHCARE LLC, WHIPPANY, NJ (US)

(72) Inventors: KANGPING XIAO, WESTFIELD, NJ (US); QIUXIA ZHAO, BERNARDSVILLE, NJ (US)

(21) Appl. No.: 16/973,543

(22) PCT Filed: Jun. 21, 2019
(86) PCT No.: PCT/US2019/038385

§ 371 Date: Dec. 9, 2020

Related U.S. Application Data
(60) Provisional application No. 62/693,086, filed on Jul. 2, 2018.

(51) Int. Cl. A61K 31/4174; A61K 47/02; A61K 47/12; A61K 47/54; A61K 9/08; A61K 9/00; A61M 15/00; A61M 15/08

(52) U.S. Cl. CPC A61K 31/4174; A61K 47/02; A61K 47/12; A61M 15/08; A61K 9/08; A61K 9/0043; A61M 15/009; A61K 47/547

(57) ABSTRACT

The present disclosure relates generally to pharmaceutical formulations of oxymetazoline and, more specifically, formulations of oxymetazoline containing one or more transition metal additives and having enhanced stability against degradation.

### CROSS-REFERENCE TO RELATED APPLICATIONS

This application claims priority to U.S. Provisional Application No. 62/693,086, filed Jul. 2, 2018, the disclosure of which is hereby incorporated by reference in its entirety.

### FIELD OF THE INVENTION

The present disclosure relates generally to pharmaceutical formulations of oxymetazoline and, more specifically, formulations of oxymetazoline comprising one or more transition metal additives and having enhanced stability against degradation.

### BACKGROUND

Oxymetazoline is a widely used over-the-counter drug for the treatment of sinus congestion. Unfortunately, oxymetazoline is highly susceptible to degradation, which reduces its storage stability and deleteriously affects the efficacy of the oxymetazoline-containing medications over time. Despite decades' of oxymetazoline use in pharmaceuticals, few formulations have been developed which manage to preserve the shelf-life of oxymetazoline medications beyond a couple of years.

As a further obstacle to the preparation of stable oxymetazoline formulations, oxymetazoline may undergo multiple degradation pathways induced by several external environmental factors—including heat, humidity, and light—as well as reactive impurities within the formulations themselves, and, thus, can produce more than one type of degradation product. Moreover, it remains unclear whether any of these undesirable degradation products are themselves entirely safe for humans, as the mutagenicity of at least one degradation product is suspected.

As such, there is a need for additional formulations of oxymetazoline which are stable over the long-term—by minimizing and/or eliminating the formation of oxymetazoline degradation products—thereby improving medication shelf-life and reducing any health risk from potential mutagenic exposure.

### SUMMARY

The present disclosure addresses this need by providing stable pharmaceutical formulations of oxymetazoline comprising one or more transition metal additives and having enhanced stability to degradation.

In one aspect, the present disclosure provides a pharmaceutical formulation, having 0.005% w/v to 0.05% w/v oxymetazoline hydrochloride, pharmaceutically acceptable excipients, and one or more transition metal additives, wherein the pharmaceutical formulation has a total transition metal concentration of at least 10 ppm.

In another aspect, provided herein is a pharmaceutical formulation, having 0.005% w/v to 0.05% w/v oxymetazoline hydrochloride, pharmaceutically acceptable excipients, and one or more transition metal additives, wherein the pharmaceutical formulation has a total transition metal concentration of at least 10 ppm, and wherein at least 75% oxymetazoline remains after the pharmaceutical formulation is subjected to controlled light exposure in accordance with ICH Photostability Testing standards in a transparent container.

In another aspect, provided herein is also a method of treating sinus congestion, comprising administering to a patient in need of treatment thereof a pharmaceutical formulation, having 0.005% w/v to 0.05% w/v oxymetazoline hydrochloride, pharmaceutically acceptable excipients, and one or more transition metal additives, wherein the pharmaceutical formulation has a total transition metal concentration of at least 10 ppm.

In still another aspect, the present disclosure provides a nasal spray system, comprising a pharmaceutical formulation comprising oxymetazoline and one or more transition metal additives as described herein, and a container containing the pharmaceutical formulation therein.

### DESCRIPTION OF THE FIGURES

FIG. 1 depicts a plot of the percentage of oxymetazoline hydrochloride remaining in pharmaceutical formulations having variable total transition metal concentrations after controlled light exposure.

FIG. 2 depicts a plot of the percentage of oxymetazoline hydrochloride remaining in pharmaceutical formulations containing different metal additives after controlled light exposure.

FIG. 3 depicts a plot of the percentage of oxymetazoline hydrochloride remaining in pharmaceutical formulations containing no additives, containing a transition metal additive, or containing both a transition metal additive and chelating agent after controlled light exposure.

FIG. 4 depicts a plot of the percentage of oxymetazoline hydrochloride remaining in pharmaceutical formulations containing no additives, containing a transition metal additive, or containing both a transition metal additive and antioxidant after controlled light exposure.

### DETAILED DESCRIPTION

Oxymetazoline is a well-known over-the-counter topical decongestant, which is typically administered as a water-based nasal spray to provide relief from sinus pressure and congestion associated with the common cold, hay fever, and upper respiratory allergies. However, despite the widespread use and success of these over-the-counter medications in treating sinus congestion, existing oxymetazoline formulations suffer from decreasing efficacy and increasing amounts of potential mutagenic degradation product (DegD) over time due to degradation of the active ingredient oxymetazoline.

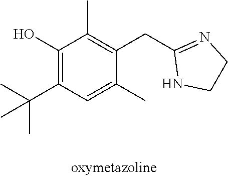 [SMILES: Cc1cc(C(C)(C)C)c(O)c(C)c1CC1=NCCN1]

Over the last few decades, researchers have attempted to develop oxymetazoline formulations which have reduced susceptibility to degradation, thereby providing more stable medications. However, a major impediment in the preparation of oxymetazoline formulations having enhanced stability is the susceptibility of oxymetazoline to, not one, but many different degradation pathways, which often leads to multiple degradation products. For example, N-(2-amino-ethyl)-2-(4-tert-butyl-3-hydroxy-2,6-dimethyl-phenyl)-acetamide (DegA), 6-tert-Butyl-3-(4,5-dihydro-1H-imidazol-2-ylmethyl)-4-hydroxy-2,4-dimethyl-cyclohexa-2,5-dienone (DegB), 2-(4-(tert-butyl)-3-hydroxy-2,6-dimethylbenzyl)-4,5-dihydro-1H-imidazole 3-oxide (DegC or Oxymetazoline N-oxide), and 6-(tert-butyl)-3-((4,5-dihydro-1H-imidazol-2-yl)methyl)-4-hydroperoxy-2,4-dimethylcyclohexa-2,5-dien-1-one (DegD) are among the principal degradation products observed in oxymetazoline formulations over time. However, DegA is largely a byproduct of hydrolytic degradation pathways, whereas DegD is a degradation product formed under photolytic stress. As such, any formulation of oxymetazoline should attenuate, if not eliminate, all reactive pathways which lead to unwanted degradation products in order to achieve the desired stability and shelf-life greater than two years.

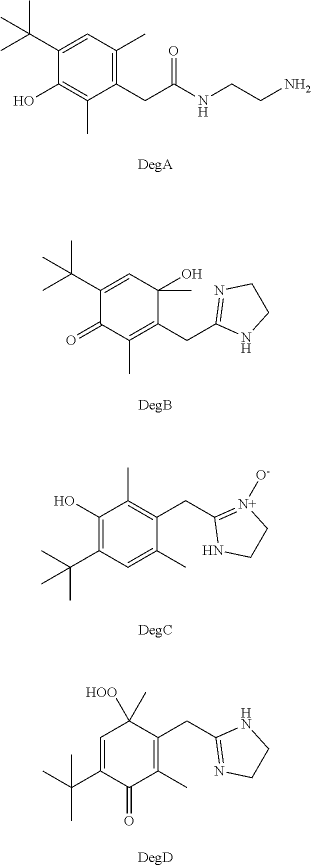

For this reason, oxymetazoline medications often combine several stabilizing additives in a multi-pronged approach to mitigate the formation of each degradation product. Existing over-the-counter oxymetazoline formulations are often specially tailored to reduce degradation through, for example, the particular selections of solvent and/or excipients, the introduction of chelating agents, use of pH-modulating buffering agents, or the addition of antioxidants, or combinations thereof. Such over-the counter oxymetazoline formulations are further sold in specialized packaging that protects the medications from variable external conditions including heat, humidity, and light, which may initiate the degradation process or, once it has begun, can accelerate it further.

Yet even with these additional packaging precautions, current formulations remain insufficient to reduce degradation adequately enough to stabilize oxymetazoline medications beyond the current average shelf-life of two years. Perhaps even more worrisome is that at least one of the known degradation products of oxymetazoline, DegD, is a suspected mutagenic compound. As such, any accumulation of degradation products in the oxymetazoline medications, particularly DegD, is still cause for concern regardless of how low the concentrations may be. The following table provides the acceptance criteria for impurities in oxymetazoline hydrochloride-containing products according to the U.S. Pharmacopeia (USP41-NF36):

| Component | Acceptance Criteria, NMT* (%) |
|---|---|
| Oxymetazoline-related compound A** | 0.15 |
| Any individual unspecified impurity | 0.1 |
| Total impurities | 0.5 |

*NMT: not more than
**DegA, N-(2-amino-ethyl)-2-(4-tert-butyl-3-hydroxy-2,6-dimethyl-phenyl)-acetamide

Thus, there remains a need to develop stable formulations of oxymetazoline that have a shelf-life greater than two years, and that mitigate oxymetazoline degradation more effectively than existing formulations. More particularly, there is a need for targeted formulations that prevent the formation of potentially harmful degradation products, such as DegD, for consumer-safe oxymetazoline medications.

Described are pharmaceutical formulations of oxymetazoline having enhanced stability against degradation. More specifically, described are oxymetazoline formulations which are especially stable to light-induced degradation pathways and thereby minimize the formation of the suspected mutagenic compound DegD.

The oxymetazoline formulations of the present disclosure achieve enhanced stability by utilizing at least one transition metal additive in the formulation. The improvement in stability is tied to the specific use of transition metal-based additives rather than any corresponding alkali or alkaline earth metal additives. It has been surprisingly found that the addition of transition metal additives at low concentrations serves to stabilize formulations comprising oxymetazoline against degradation. In particular, the use of transition metal additive above a threshold amount significantly reduces the formation of photo-degradation product DegD, in some cases to negligible or non-detectable levels, as well as minimizing the formation of other major degradation products, such as DegA and DegB. The reductions in the formation of such degradation products can be assessed under controlled stress testing, including but not limited to controlled light exposure and elevated temperatures as described herein.

The formulations of the present disclosure comprising oxymetazoline and one or more transition metal additives may be further combined with other stabilizing agents—such as chelating agents, antioxidants, and buffering agents—to augment the effect of the transition metal additives in preventing the formation of DegD or to provide complementary stability against other degradation pathways. In summary, the present disclosure provides for pharmaceutical formulations, which allow for oxymetazoline medications having enhanced stability against degradation and which could lead to oxymetazoline-based medications having prolonged shelf-lives greater than two years even without the use of specialized packaging.

The following description sets forth exemplary methods, parameters and the like. It should be recognized, however, that such description is not intended as a limitation on the scope of the present disclosure but is instead provided as a description of exemplary embodiments.

Reference to “about” a value or parameter herein includes (and describes) embodiments that are directed to that value or parameter per se. For example, description referring to “about X” includes description of “X”.

It is understood that aspects and variations described herein also include “consisting” and/or “consisting essentially of” aspects and variations.

### Pharmaceutical Formulations of Oxymetazoline

In one aspect, provided herein is a pharmaceutical formulation comprising oxymetazoline hydrochloride and one or more transition metal additives.

Oxymetazoline is used extensively in over-the-counter medications to treat, for example, sinus congestion and pressure. In existing pharmaceutical formulations of oxymetazoline, such as nasal sprays, the concentration of oxymetazoline required to provide the desired decongestant effect is quite low. Nasal spray formulations currently being sold usually contain oxymetazoline as its hydrochloride salt at concentration of about 0.05% weight/volume (w/v). In some embodiments, provided herein are pharmaceutical formulations comprising 0.05% w/v oxymetazoline hydrochloride. It should be recognized that the formulations described herein may also be suitable for use in applications requiring lower active concentrations of oxymetazoline, such as ophthalmic solutions. In other embodiments, the formulations described herein may have as low as 0.005% w/v oxymetazoline hydrochloride. In certain embodiments, the pharmaceutical formulations described in the present disclosure comprise 0.005% w/v to 0.05% w/v oxymetazoline hydrochloride. Due to the low concentration of oxymetazoline in these formulations, even minuscule rates of degradation over several degradation pathways are likely to have a large impact on the efficacy of the medication with time.

In fact, the concentration of oxymetazoline of currently marketed medications is often on the same order of magnitude with that of reactive impurities present in such formulations. Reactive impurities, including residual heavy metal catalysts and free radical initiators, may be introduced into the pharmaceutical formulations through added polymeric excipients, such as polyethylene glycol and povidone, which often constitute the second and third largest components of oxymetazoline formulations other than purified water. As such, it is difficult to reduce the concentration of reactive impurities by reducing the excipient content without also compromising the properties of the overall formulation.

Rather than remove excipients to reduce the amount of reactive impurities, existing formulations of oxymetazoline often utilize chelating agents and antioxidants to sequester reactive impurities and inhibit reactions of said impurities with oxymetazoline. Buffering agents to control the pH of such formulations may also be added to make certain oxymetazoline degradation pathways less energetically favorable. However, these additives alone remain insufficient to eliminate all degradation pathways of oxymetazoline and the resulting formulations have shelf-lives of about two years at best. Indeed, the observation that sequestration or inhibition of the reactive impurities alone would not necessarily ensure a longer shelf-life for oxymetazoline medications is not wholly unexpected, as reactive impurities are not the only cause of oxymetazoline degradation.

### Transition Metal Additives

It has been surprisingly found that the introduction of transition metal additives to pharmaceutical formulations of oxymetazoline at low concentrations confers improved stability to oxymetazoline against degradation and, as a result, a prolonged shelf-life to the formulations. The surprising effect of this addition is in part due to the fact that trace amounts of metals in pharmaceutical formulations are commonly viewed in the art as reactive impurities to be removed by chelating agents as described above. Yet, by incorporating at least one transition metal additive to provide transition metal content above a threshold concentration, the oxymetazoline formulations of the present disclosure achieve enhanced stability against degradation, specifically photo-degradation. It should be recognized that the oxymetazoline formulations may contain more than one transition metal additive. In some embodiments, the pharmaceutical formulations as described herein comprise one or more transition metal additives. In other embodiments, the pharmaceutical formulations described herein comprise two or more transition metal additives.

As noted above, the transition metal additives of the present disclosure provide a stabilizing effect against photo-degradation above a threshold concentration. In fact, the key factor in achieving the observed stability of oxymetazoline against degradation appears to be the total transition metal concentration. The total transition metal concentration is equal to the sum of the transition metal concentrations afforded by each individual transition metal additive in the pharmaceutical formulation. Below a certain level, the total transition metal concentration may be insufficient to inhibit the degradation of oxymetazoline. However, beyond a certain concentration, increasing amounts transition metal additives are unlikely to add any further stabilizing benefit to the pharmaceutical formulations described herein, and may even approach harmful levels for human intake. As such, the total transition metal concentration is carefully controlled so that the need for adequate levels of the transition metal additives to achieve the desired formulation stability is balanced with considerations of manufacturing cost and potential health risks associated with excess intake of transition metals.

In one aspect, provided herein is a pharmaceutical formulation comprising oxymetazoline hydrochloride and one or more transition metal additives, wherein the pharmaceutical concentration has a total transition metal concentration. In some embodiments, the pharmaceutical formulation has a total transition metal concentration of at least about 4 ppm, at least about 5 ppm, at least about 10 ppm, at least about 20 ppm, at least about 25 ppm, at least about 30 ppm, at least about 40 ppm, at least about 50 ppm, or at least about 100 ppm. In certain embodiments, the pharmaceutical formulation has a total transition metal concentration of at least about 25 ppm, at least about 40 ppm, or at least about 50 ppm. In other embodiments, the formulation has a total transition metal concentration of less than about 500 ppm, or less than about 100 ppm. In certain embodiments, the pharmaceutical formulation has a total transition metal concentration of between about 10 ppm and about 500 ppm, between about 10 ppm and about 200 ppm, between about 10 ppm and about 100 ppm, between about 25 ppm and about 500 ppm, between about 25 ppm and about 200 ppm, between about 25 ppm and about 100 ppm, between about 50 ppm and about 500 ppm, between about 50 ppm and about 200 ppm, or between about 50 ppm and about 100 ppm. In yet other embodiments, the pharmaceutical formulation has a total transition metal concentration of about 40 ppm, about 50 ppm, or about 100 ppm.

As with the overall concentration of transition metals in present pharmaceutical formulations, also relevant are the transition metal elements used in the transition metal additives. The transition metal additives of the present disclosure confer surprising photostability to oxymetazoline formulations which is not achieved through similar addition of alkali metal- or alkaline earth metal-based additives. For example, magnesium and calcium, both alkaline earth metals, may not be observed to provide the same photostability benefit as iron, copper or zinc. However, it should be recognized that not all transition metal elements may be suitable for use in the present pharmaceutical formulations. The selection of transition metals suitable for use in the present formulations is guided not only by the observed effectiveness of such transition metals in mitigating oxymetazoline degradation but also their cost, their abundance, and, above all else, their non-toxicity.

In some embodiments, the transition metal additive comprises a first-row transition metal. In some embodiments, the transition metal additive comprises a transition metal selected from the group consisting of titanium, manganese, iron, cobalt, copper, and zinc. In certain embodiments wherein the pharmaceutical formulation comprises one or more transition metal additive, the transition metals of each transition metal additive may be the same or different. For example, in some embodiments wherein the pharmaceutical formulation comprises one or more transition metal additive, at least one of the one or more transition metal additives comprises a transition metal selected from the group consisting of titanium, manganese, iron, cobalt, copper, and zinc. In certain embodiments, at least one of the one or more transition metal additives comprises iron, copper, or zinc. In other embodiments, at least one of the one or more transition metal additives comprises iron. It should be further recognized that the transition metal additives as described herein may contain the aforementioned transition metals in any of their oxidation states, especially oxidation states which are stable in the formulation.

The transition metal additives of the present disclosure may be provided in the form of pharmaceutically acceptable salts of the transition metals described herein. For example, pharmaceutically acceptable salts known in the art, including but not limited to sulfate, chloride, or gluconate salts, may be used. The recitation of transition metal salts to be used as transition metal additives as described herein is not intended to be limiting. It should be noted, however, that the solubility of the salts used as transition metal additives may be relevant to ensure the transition metal additive is fully incorporated, or dissolved, into the final formulation to provide the enhanced stability properties as described herein. Moreover, it is desirable that one or more transition metal additives do not interfere with the desired physical properties of the resulting formulation, such as aerosolizability in nasal sprays. Therefore, both the safety of the additives for human use and their compatibility with the formulation should be considered in identifying suitable salts to use as transition metal additives.

In some embodiments, at least one of the one or more transition metal additives comprises a sulfate, chloride, or gluconate salt. In certain embodiments, wherein at least one of the one or more transition metal additives comprises iron, the one or more transition metal additives comprise iron sulfate, iron chloride, or iron gluconate. In other embodiments, wherein at least one of the one or more transition metal additives comprises zinc, the one or more transition metal additives comprise zinc sulfate or zinc chloride. In other embodiments, wherein at least one of the one or more transition metal additives comprises copper, the one or more transition metal additives comprise copper sulfate or copper chloride. In yet another embodiment, wherein at least one of the one or more transition metal additives comprises cobalt, the one or more transition metal additives comprise cobalt sulfate or cobalt chloride. In still yet another embodiment, wherein at least one of the one or more transition metal additives comprises manganese, the one or more transition metal additives comprises manganese sulfate or manganese chloride.

### Additional Stabilizing Agents

Although the use of transition metal additives as disclosed above significantly reduces degradation of oxymetazoline and improves the stability of oxymetazoline-containing pharmaceutical formulations, combinations of other additives, such as chelating agents, antioxidants and buffering agents, may be further added to the pharmaceutical formulations. These additional chelating agents, antioxidants, and buffering agents may be useful to augment the effect of the transition metal additives in stabilizing the formulations against photo-degradation or to mitigate other degradation pathways accessible to oxymetazoline that are not fully attenuated by the transition metal additives. In some embodiments, the pharmaceutical formulations as described herein further comprise chelating agents, antioxidants, and/or buffering agents.

Chelating agents are often incorporated into pharmaceutical formulations to bind unwanted heavy metal impurities and to act as preservative. In some embodiments, the pharmaceutical formulations described herein further comprise a chelating agent. Ethylenediaminetetraacetic acid, or its conjugate base ethylenediaminetetraacetate salt or edetate salt (EDTA), is a common chelating agent, which may be used in the present pharmaceutical formulations. In certain embodiments, the chelating agent is an ethylenediaminetetraacetate salt. In yet other embodiments, the chelating agent is disodium EDTA or calcium disodium EDTA. Typically, small concentrations of chelating agents are used to provide the desired chelating or preservative effect. In some embodiments, the pharmaceutical formulation comprises 0.01% w/v EDTA. In other embodiments, the pharmaceutical formulation comprises 0.1% w/v EDTA.

Antioxidants may also be added to the pharmaceutical formulations described herein. Antioxidants are utilized to capture free radicals and other reactive impurities, which may be present in the formulations at low concentrations. In some embodiments, the pharmaceutical formulation comprises an antioxidant. In certain embodiments, the pharmaceutical formulation comprises sodium metabisulfite (Na₂S₂O₅), ascorbic acid (vitamin C), or propyl gallate (propyl 3,4,5-trihydroxybenzoate) as antioxidants. Similar to the chelating agents above, minimal concentrations of antioxidants are often used to achieve the desired reduction of free radicals and reactive impurities. In some embodiments, the pharmaceutical formulation comprises about 0.004% w/v or about 0.006% w/v antioxidant. In certain embodiments, the pharmaceutical formulation comprises about 0.004% w/v or about 0.006% w/v Na₂S₂O₅.

The pharmaceutical formulations of the present disclosure may be further modified to control the acidity of the formulation and, thus also, the reactive environment for oxymetazoline, as high levels of acidity may inhibit certain hydrolytic degradation pathways that might otherwise be prominent in neutral or basic aqueous solution. As such, in addition to the use of transition metal additives to stabilize pharmaceutical formulations, the pH of the pharmaceutical formulations may also be adjusted to minimize oxymetazoline degradation. In some embodiments, the pH of the pharmaceutical formulation is between about pH 3.00 and about pH 6.00, or between about pH 4.00 and about pH 5.00. In other embodiments, the pH of the pharmaceutical formulation is about pH 4.76.

Control over the acidity of the present oxymetazoline pharmaceutical formulations may be achieved by adding buffering agents. In some embodiments, the pharmaceutical formulations described herein comprise one or more buffering agents. In certain embodiments, the one or more buffering agents are selected from the group consisting of acetic acid, an acetate salt, citric acid, a citrate salt, phosphoric acid, a hydrogen phosphate salt, and a dihydrogen phosphate salt, and any combinations thereof. In other embodiments, the one or more buffering agents comprises citric acid, a citrate salt, phosphoric acid, or a phosphate salt, or any combinations thereof. In certain embodiments, the one or more buffering agents comprise a combination of citric acid and a phosphate salt. It should be recognized that the phosphate salt may be a monobasic or dibasic phosphate salt. In other embodiments, the one or more buffering agents comprise a combination of citric acid and disodium phosphate. In other embodiments, the one or more buffering agents comprise a combination of sodium phosphate dibasic and sodium phosphate monobasic.

The concentration of the one or more buffering agents may be tailored depending on the particular strength of each buffering agent so that the desired formulation pH is achieved as described above. In some embodiments, the total concentration of buffering agents is sufficient such that the pharmaceutical formulation has a pH of between about pH 3.00 and about pH 6.00 or between about pH 4.00 and about pH 5.00. In other embodiments, the total concentration of buffering agents in the pharmaceutical formulation is less than about 0.6% w/v. In the case of multiple buffering agents, the concentration of each individual buffering agent may be described. For example, in some embodiments, the pharmaceutical formulation comprises about 0.268% w/v citric acid and 0.313% w/v disodium phosphate, anhydrous.

### Excipients and Other Ingredients

Oxymetazoline-containing nasal sprays are typically used to provide immediate relief from sinus congestion and pressure. Immediate relief from such symptoms is achieved through nasal administration and direct absorption of oxymetazoline through the affected mucous membranes of the nasal cavity. In addition to the stabilizing additives described above, which are included in the present pharmaceutical formulations to mitigate the multiple degradation pathways of oxymetazoline, the pharmaceutical formulations may also comprise any pharmaceutically acceptable excipients, dispersants, or diluents to give the final oxymetazoline formulations the desired physical properties for nasal administration.

For applications of oxymetazoline to provide immediate relief from sinus congestion and pressure, the aerosolizability of the formulation is a key parameter to ensure that it may be administered as a nasal spray. As previously noted, oxymetazoline is typically utilized in the form of its hydrochloride salt for pharmaceutical formulations. The hydrochloride salt of oxymetazoline is reasonably soluble in water and water is readily aerosolized. As such, water may be used as the primary excipient, or vehicle, to deliver oxymetazoline in aerosol form. In some embodiments, the pharmaceutical formulations comprise water. In some embodiments, the pharmaceutical formulations are aqueous.

As the primary excipient, the quantity of water used in the pharmaceutical formulations described herein is relevant insofar as sufficient water is added to achieve both the necessary aerosolizability for the formulation and the desired concentrations of the oxymetazoline, the transition metal additives, and any additional stabilizing additives disclosed above, as well as any other excipients or ingredients disclosed below. In some embodiments, the pharmaceutical formulation comprises at least about 80% w/v water, at least about 85% w/v water, or at least about 87% w/v water.

Other excipients may be included in the pharmaceutical formulations described herein to ensure that the oxymetazoline, transition metal additives, and any other stabilizing additives—all of which are typically present in minute concentrations less than 1% w/v—are evenly distributed throughout the aqueous formulation. Moreover, additional excipients may be used to adjust the physical properties of the aqueous formulation, for example, to modulate viscosity to facilitate nasal administration. In some embodiments, the pharmaceutical formulations of oxymetazoline herein may comprise polyethylene glycol, povidone, and a mixture microcrystalline cellulose and sodium carboxymethylcellulose.

Minimal quantities of the non-water excipients may be used in the present pharmaceutical formulations. Indeed, small concentrations of excipients such as polyethylene glycol, povidone, and a mixture of microcrystalline cellulose and sodium carboxymethylcellulose are typically sufficient to achieve the desired dispersion and solubilization of oxymetazoline, the transition metal additives and other stabilizing agents, largely because these components to be dissolved are present in such low concentrations themselves. However, using small concentrations of these other excipients is also advantageous to minimize the introduction of unwanted heavy metals or reactive impurities into the formulation, which might otherwise detract from the stabilizing effects achieved by the transition metal additives, chelating agents, antioxidants, and buffering agents described above.

Polyethylene glycol may be used to aid dispersion of the active pharmaceutical ingredient, transition metal additives and other stabilizing agents in the pharmaceutical formulations described herein. Polyethylene glycol may be identified by other common synonyms known in the art including but not limited to Macrogol and/or PEG. In some embodiments, the pharmaceutical formulation comprises polyethylene glycol. It should be noted that particular grades of polyethylene glycol, defined by weight average molecular weight, for example, may be especially useful for the pharmaceutical formulations of the present disclosure. In some embodiments of the foregoing, the pharmaceutical formulation comprises polyethylene glycol, wherein the polyethylene glycol has a weight average molecular weight between about 1,300 and about 1,600 g/mol.

As disclosed above, the concentration of polyethylene glycol to be used in the pharmaceutical formulations herein is adjusted carefully to ensure proper dispersion of the oxymetazoline, transition metal additives, and other stabilizing agents in the aqueous formulation, without interfering with the physical properties of the oxymetazoline formulation. In certain embodiments, the pharmaceutical formulation comprises about 5% w/v polyethylene glycol.

As an additional excipient, povidone—also known as polyvinylpyrrolidone or PVP, or other registered names including Kollidon®—may be incorporated into the present pharmaceutical formulations as a solubilizing agent for the active pharmaceutical ingredient, transition metal additives and other stabilizing agents, as well as to modify the physical properties of the formulation as desired. In some embodiments, the pharmaceutical formulation comprises polyvinylpyrrolidone, or povidone or PVP. Moreover, various grades of povidone may be utilized as excipients in the present formulations although certain grades may be preferred. Different grades of povidone may be defined according to, for example, weight average molecular weight, viscosity average molecular weight and/or K-value. In certain embodiments, the pharmaceutical formulation comprises povidone having an average K-value between 29 and 32.

As with polyethylene glycol, the concentration of povidone to be used in the present formulations should be sufficient enough to provide the desired solubilizing effect without interfering with the desired physical properties of the formulation. In certain embodiments, the pharmaceutical formulation comprises 3% w/v povidone.

Similar to polyethylene glycol and povidone above, the pharmaceutical formulation may further comprise a mixture of microcrystalline cellulose and carboxymethylcellulose sodium (also known as carmellose sodium) to modulate the physical properties of the oxymetazoline formulation, such as viscosity and aerosolizability, and to aid dispersion of oxymetazoline, the transition metal additives, and other stabilizing agents. Mixtures of microcrystalline cellulose and carmellose sodium are also known in the art as colloidal microcrystalline cellulose or dispersible microcrystalline cellulose, as well as by a variety of registered names including Avicel®. In some embodiments, the pharmaceutical formulation comprises a mixture of microcrystalline cellulose and carboxymethylcellulose sodium.

The concentration of the mixture of microcrystalline cellulose and carmellose sodium present in the pharmaceutical formulation may be small but sufficient enough to provide the desired physical properties to the formulation but without detracting from the stability of the formulation provided by the transition metal additives and other stabilizing agents. In certain embodiments, the pharmaceutical formulation comprises 3% w/v a mixture of microcrystalline cellulose and carboxymethylcellulose sodium.

Other agents may be added to further preserve the pharmaceutical formulations disclosed herein, for example, by inhibiting unwanted biological growth, or to improve palatability of the medication for the consumer. In some embodiments, the pharmaceutical formulation comprises preservatives to inhibit unwanted biological growth. In certain embodiments, the pharmaceutical formulation comprises benzalkonium chloride. In still yet other embodiments, the pharmaceutical formulation comprises flavorants.

### Assessing Pharmaceutical Formulation Stability

Provided herein are pharmaceutical formulations of oxymetazoline having enhanced stability, particularly with respect to photo-degradation, as compared to existing oxymetazoline medications on the market. The improved stability of these oxymetazoline formulations is achieved through the use of one or more transition metal additives, which reduce the formation unwanted degradation products produced from several different reactive pathways.

### Conditions for Stability Assessment

The improved stability of the present pharmaceutical formulations can be assessed under a variety of conditions as described herein. For example, the stability of the pharmaceutical formulations of oxymetazoline may be assessed under normal storage conditions, such as under dry, dark conditions at controlled room temperature (20° C. to 25° C.). Alternatively, the stability of the pharmaceutical formulations described herein may be assessed under applied external stressors, such as elevated temperatures, increased humidity, or controlled concentrated light exposure, intended to simulate extreme environmental conditions and accelerate degradation for analysis on a practicable timescale in a laboratory setting. It is useful to specify the conditions under which the stability of the present pharmaceutical formulations is evaluated, particularly in view of the many degradation pathways of oxymetazoline, each of which may be preferentially initiated under different conditions.

For example, as noted above, the formation of the degradation product 6-(tert-butyl)-3-((4,5-dihydro-1H-imidazol-2-yl)methyl)-4-hydroperoxy-2,4-dimethylcyclohexa-2,5-dien-1-one, or DegD, is a largely light-initiated process. Moreover, the use of transition metal additives in the formulations of the present disclosure is targeted to minimize the formation of this potentially mutagenic compound DegD. As such, the stability of the present oxymetazoline formulations comprising one or more transition metal additives and the formation of DegD may be examined under photostability stress tests. The sensitivity of the present pharmaceutical formulations to photo-degradation may be assessed under controlled light exposure, using a light source having a well-defined spectral profile and power output per unit area, for a specified duration of time. In some embodiments, the pharmaceutical formulations described herein are subjected to controlled light exposure.

The International Conference on Harmonisation of Technical Requirements for Registration of Pharmaceuticals for Human Use (ICH) “Photostability Testing of New Drug Substances and Products Q1B”, established on November 1996, provides standards for suitable light sources and evaluation procedures, which may be used to gauge photo-degradation in the pharmaceutical formulations of the present disclosure. In other embodiments, the pharmaceutical formulations described herein are subjected to controlled light exposure in accordance with ICH Photostability Testing standards. For instance, according to the ICH Photostability Testing standards, the pharmaceutical formulations described herein may be exposed to a light source meeting the standard spectral output of Option 1 or 2 in the table below, or any other equivalents thereof, for a time period sufficient to provide a total illumination of at least 1.2 million lux-hours of both visible and near ultraviolet light, and for which the near ultraviolet light has an energy intensity of at least 200 watt-hours per square meter.

Light Sources for ICH Photostability Testing

| Option | Light Characteristics |
|---|---|
| 1 | Any light source designed to produce output similar to D65/ID65 emission |
| | standard such as an artificial daylight fluorescent lamp combining visible and |
| | ultraviolet (UV) outputs, xenon, or metal halide lamp.* |
| 2 | The same sample should be exposed to both the cool white fluorescent and |
| | near UV lamp: |
| | 2.1. Cool, white fluorescent lamp designed to produce an output similar to |
| | that ISO 10977 (1993); and |
| | 2.2. A near UV fluorescent lamp having a spectral distribution from 320 nm |
| | to 400 nm with a maximum energy emission between 350 nm and 370 nm** |

*D65 is the internationally recognized standard for outdoor daylight as defined in ISO 10977 (1993). ID65 is the equivalent indoor indirect daylight standard. For a light source emitting significant radiation below 320 nm, an appropriate filter(s) may be fitted to eliminate such radiation
**a significant proportion of UV should be in both bands of 320-360 nm and 360-400 nm.

As acknowledged above, currently marked oxymetazoline medications are sold in specialized packaging intended to isolate the medications from variable external conditions, including light exposure. It is useful to assess the photostability of the present pharmaceutical formulations with and without such specialized packaging to differentiate the stabilizing effects provided directly by the transition metal additives and other stabilizing agents of the formulation from any additional protective effects provided by special packaging. The ICH Photostability Testing guidelines also provide for evaluation of drug substances and drug products under progressively increasing levels of packaging—that is, from direct exposure of drug substance and/or product alone, to exposure of the drug substance and/or product in immediate packaging, to exposure of the drug substance and/or product in immediate packaging and any secondary cartons. To assess whether the addition of transition metal additives renders such specialized packaging superfluous, the photostability of the present pharmaceutical formulations may be evaluated in containers which are either opaque to visible and/or ultraviolet light, transparent to all visible and/or ultraviolet light, or transparent to select wavelengths of visible and/or ultraviolet light. In some embodiments of the foregoing, the pharmaceutical formulation is subjected to controlled light exposure in accordance with ICH Photostability Testing standards, wherein the pharmaceutical formulation is contained in an opaque container. In other embodiments of the foregoing, the pharmaceutical formulation is contained in a transparent container, such as a clear glass bottle. In other embodiments, pharmaceutical formulation is contained in an opaque container. In still yet other embodiments, the container is opaque to ultraviolet light, such as a brown bottle.

In addition to photostability stress testing to evaluate formation of suspected mutagenic DegD in the present pharmaceutical formulations, other stress testing methods known in the art can be used to assess and quantify the formation of other known degradation products, such as DegA, DegB, and DegC. For example, DegA is a major degradation product of oxymetazoline which is principally formed via a temperature- and water-dependent degradation pathways. As such, the sensitivity of oxymetazoline to heat-induced degradation may be evaluated by subjecting the pharmaceutical formulations of the present disclosure to elevated temperatures for a period of time. In some embodiments, the pharmaceutical formulations described herein are subjected to elevated temperatures, such as at least about 70° C. or at least about 75° C. for a specified period of time, for example at least about 1 day, at least about 3 days, at least about 5 days, at least about 7 days, at least about 10 days, at least about 12 days or at least about 14 days. In certain embodiments, the pharmaceutical formulations are subjected to an elevated temperature of 75° C. for about 14 days.

### Stability Metrics

The incorporation of transition metal additives into the pharmaceutical formulations of the present confers improved stability of oxymetazoline against degradation and, thus, allows for formulations having prolonged shelf-lives. In addition to the various stress testing conditions known in the art for evaluating the stability of the present pharmaceutical formulations, the stability itself may be also characterized by several metrics as well.

A common metric for assessing the stability of over-the-counter medications is the shelf-life of such medications. The shelf-life may be described as the length of time during which a drug substance or product remains generally with its approved specifications for safety and therapeutic efficacy. The present pharmaceutical formulations as described herein may be similarly assessed. For example, in some embodiments, provided herein is a pharmaceutical formulation comprising oxymetazoline and one or more transition metal additives, wherein the pharmaceutical formulation has a shelf-life of at least about 24 months, at least about 30 months, at least about 36 months, at least about 42 months, at least about 48 months, at least about 54 months, or at least about 60 months. In certain embodiments, the pharmaceutical formulation has a shelf-life of at least about 24 months. It is expected that evaluation of the shelf-life is conducted under normal storage conditions, as defined above, unless otherwise noted.

As related to the shelf-life, it may be useful to further characterize the compositional purity of the pharmaceutical formulations described herein to determine whether the formulations remain within their specified safety and efficacy ranges based on the concentrations of oxymetazoline and/or any degradation products. However, assessment of compositional purity is also an effective metric to characterize the stability of the present formulations under applied environmental stressors, such as controlled light exposure and elevated temperatures as disclosed above.

The stability of the oxymetazoline formulations as described herein may be characterized and compared to existing formulations with respect to the quantity of oxymetazoline that remains intact and/or has degraded in the formulation after exposure to any of the aforementioned environmental conditions. In order to quantify the amount of oxymetazoline that either remains intact or has degraded, the remaining and degraded oxymetazoline may be calculated as percentages of the original oxymetazoline concentration in the formulation as prepared. For example, the original oxymetazoline concentration in the formulation may be taken as the concentration prior to any period of time stored under normal storage conditions, prior to any controlled light exposure, or prior to exposure to elevated temperatures. The oxymetazoline remaining in the pharmaceutical formulation after being subjected to a stress test may be determined, for example, by HPLC characterization against a chemical standard and/or known quantity of oxymetazoline to determine absolute content of oxymetazoline, which may then be converted to a percentage of the original oxymetazoline concentration. The quantity of oxymetazoline degraded may then be calculated as the difference of the percentage of oxymetazoline remaining in the formulation after exposure to test conditions and the percentage of oxymetazoline present (100%) in the original formulation.

In some embodiments, the pharmaceutical formulations of the present disclosure comprise oxymetazoline and one or more transition metal additives, wherein at least about 75%, at least about 80%, at least about 85%, at least about 90%, at least about 95%, or at least about 99% oxymetazoline remains after at least 24 months of storage under normal storage conditions. In other embodiments, the pharmaceutical formulation comprises oxymetazoline and one or more transition metal additives, wherein at least about 75%, at least about 80%, at least about 85%, at least about 90%, at least about 95%, or at least about 99% oxymetazoline remains after the pharmaceutical formulation is subjected to controlled light exposure in accordance with ICH Photostability Testing standards. In still yet other embodiments, the pharmaceutical formulation comprises oxymetazoline and one or more transition metal additives, wherein at least about 75%, at least about 80%, at least about 85%, at least about 90%, at least about 95%, or at least about 99% oxymetazoline remains after the pharmaceutical formulation is subjected to an elevated temperature of 75° C. for 14 days or longer.

In other embodiments of the present pharmaceutical formulations, wherein the quantity of oxymetazoline that has degraded is assessed, less than about 25%, less than about 20%, less than about 15%, less than about 10%, less than about 5%, or less than about 1% oxymetazoline has degraded after at least 24 months of storage under normal storage conditions. In other embodiments, the pharmaceutical formulation comprises oxymetazoline and one or more transition metal additives, wherein less than about 25%, less than about 20%, less than about 15%, less than about 10%, less than about 5%, or less than about 1% oxymetazoline has degraded after the pharmaceutical formulation is subjected to controlled light exposure in accordance with ICH Photostability Testing standards. In still yet other embodiments, the pharmaceutical formulation comprises oxymetazoline and one or more transition metal additives, wherein less than about 25%, less than about 20%, less than about 15%, less than about 10%, less than about 5%, or less than about 1% oxymetazoline has degraded after the pharmaceutical formulation is subjected to an elevated temperature of 75° C. for 14 days or longer.

However, it should be recognized that the amount of oxymetazoline remaining or degraded in the pharmaceutical formulation is only a part of the overall consideration for an acceptable shelf-life. As described above, oxymetazoline is susceptible to multiple degradation pathways and thereby can produce multiple degradation products, some of which may be more or less prevalent than others and some of which may pose potential, unique health concerns to the consumer. As such, both the identities and quantities of the multiple degradation products may also be determined. It is especially useful to assess the quantities of each degradation product present in the formulations so as to characterize whether the formulations have only lost their therapeutic efficacy and drug safety due to oxymetazoline degradation or, from a potentially worse standpoint, whether they have also accumulated potentially mutagenic degradation products such as DegD and should no longer be administered. The stability of the pharmaceutical formulations may be characterized by the quantities of each degradation product individually—DegA, DegB, DegC, DegD, or considered as particular combinations of degradation products at certain weight volume percentages or concentrations in part-per-million. It should also be noted that different degradation test conditions are expected to result in different composition profiles for the formulations—that is, the presence/absence of certain degradation products and/or differing concentrations of said products. For example, controlled light exposure may produce a different profile of degradation products or concentrations of degradation products as compared to the profile obtained under elevated temperatures.

In some embodiments, the pharmaceutical formulation comprises less than about 0.1% w/v DegD after the pharmaceutical formulation is subjected to controlled light exposure in accordance with ICH Photostability Testing standards. In other embodiments, the pharmaceutical formulation comprises less than about 0.1% w/v DegA after the pharmaceutical formulation is subjected to controlled light exposure in accordance with ICH Photostability Testing standards. In still yet other embodiments, the pharmaceutical formulation comprises less than about 0.1% w/v DegB after the pharmaceutical formulation is subjected to controlled light exposure in accordance with ICH Photostability Testing standards. In certain embodiments, the pharmaceutical formulation comprises: (i) less than about 0.1% w/v DegD, (ii) less than about 0.15% w/v DegA, or (iii) less than about 0.1% w/v DegB, or any combinations thereof after the pharmaceutical formulation is subjected to controlled light exposure in accordance with ICH Photostability Testing standards. In some embodiments, the pharmaceutical formulation comprises less than 0.5% w/v total impurities after the pharmaceutical formulation is subjected to controlled light exposure in accordance with ICH Photostability Testing standards. In still yet other embodiments, the pharmaceutical composition may comprise a non-detectable amount of DegD, DegA, or DegB, or any combinations thereof, after the pharmaceutical formulation is subjected to controlled light exposure in accordance with ICH Photostability Testing standards.

In yet other embodiments, the pharmaceutical formulation comprises less than about 0.1% w/v DegD after the pharmaceutical formulation is subjected to elevated temperature of about 75° C. for at least about 14 days. In other embodiments, the pharmaceutical formulation comprises less than about 0.15% w/v DegA after the pharmaceutical formulation is subjected to elevated temperature of about 75° C. for at least about 14 days. In still yet other embodiments, the pharmaceutical formulation comprises less than about 0.1% w/v DegB after the pharmaceutical formulation is subjected to elevated temperature of about 75° C. for at least about 14 days. In certain embodiments, the pharmaceutical formulation comprises: (i) less than about 0.1% w/v DegD, (ii) less than about 0.15% w/v DegA, or (iii) less than about 0.1% w/v DegB, or any combinations thereof after the pharmaceutical formulation is subjected to elevated temperature of about 75° C. for at least about 14 days. In some embodiments, the pharmaceutical formulation comprises less than 0.5% w/v total impurities after the pharmaceutical formulation is subjected to elevated temperature of about 75° C. for at least about 14 days. In still yet other embodiments, the pharmaceutical composition may comprise a non-detectable amount of DegD, DegA, or DegB, or any combinations thereof, after the pharmaceutical formulation is subjected to elevated temperature of about 75° C. for at least about 14 days.

### Methods of Administration and Nasal Spray Systems for Storage and Administration

Oxymetazoline is a topical decongestant used to treat sinus congestion and pressure associated with the common cold, hay fever, and upper respiratory allergies. Unlike other common decongestants such as pseudoephedrine or phenylephrine, which are ingested orally and are delayed in effect, oxymetazoline-containing medications are typically administered directly into the nostrils as a spray to provide immediate relief from nasal congestion. As such, the present disclosure also provides for methods of administering the pharmaceutical formulations comprising oxymetazoline and one or more transition metal additives for the treatment and/or relief of sinus congestion and pressure.

In one aspect, provided herein is a method for treating sinus congestion and pressure comprising administering a pharmaceutical formulation comprising oxymetazoline hydrochloride and one or more transition metal additives to a patient in need of treatment thereof. In certain embodiments, the method comprises administering the pharmaceutical formulation via nasal administration. In yet other embodiments, the method comprises administering the pharmaceutical formulation as a nasal spray.

In another aspect, provided herein are also nasal spray systems comprising the pharmaceutical formulations of the present disclosure. The nasal spray systems of the present disclosure serve as both a storage container for the oxymetazoline formulations described herein and as a means for administration of the formulations directly from the storage container to the affected nasal passages. In some embodiments, the nasal spray system comprises a pharmaceutical formulation comprising oxymetazoline hydrochloride and one or more transition metal additives, and a container containing the pharmaceutical formulation therein. In some embodiments, the container is a glass or plastic bottle. In certain embodiments, the nasal spray system further comprises a pump, wherein the pump is attached to the container and is configured to aerosolize the pharmaceutical formulation. In other embodiments, the system comprises a nozzle, wherein the nozzle is attached to the pump and is configured to receive the aerosolized pharmaceutical formulation and to deliver the aerosolized pharmaceutical formulation into a nostril or a nasal cavity.

Oxymetazoline medications currently on the market are typically packaged in special containers for the purpose of minimizing exposure of the formulations to light, changes in temperature and variations in humidity. Similarly, the pharmaceutical formulations described herein may be specially packaged to protect oxymetazoline from degradation. In some embodiments of the foregoing, the nasal spray system comprises a container that is opaque to light. In other embodiments, the nasal spray system comprises a container that is opaque to certain wavelengths of light. In certain embodiments, the nasal spray system comprises a container that is opaque to visible and/or ultraviolet light. In other embodiments, the container does not transmit visible and/or ultraviolet light. In certain embodiments, the container is opaque to ultraviolet light.

It should be recognized, however, that with the addition of transition metal additives to the present pharmaceutical formulations, photo-induced degradation of oxymetazoline may be mitigated to such an extent that light-blocking properties of the specialized packaging are no longer necessary to provide oxymetazoline, and thus also formulation, photostability. As such, the specialized packaging may still be useful for isolating the present pharmaceutical formulations from fluctuations in temperature and humidity and to facilitate administration of oxymetazoline as a nasal spray, but may be rendered otherwise unnecessary for light protection. Thus, in some embodiments, the container is transparent to all visible and/or ultraviolet light. In certain embodiments, the container transmits visible and/or ultraviolet light. In other embodiments, the container is transparent to select wavelengths of visible and/or ultraviolet light. In certain embodiments, the container is transparent to ultraviolet light.

### EXAMPLES

### Example 1: Transition Metal Additive Reduction of Photolytic Degradation

Preparation of Oxymetazoline Basic Formulation. A basic oxymetazoline formulation was prepared by combining Avicel® RC 591 (microcrystalline cellulose and carmellose sodium), povidone K29-32, polyethylene glycol 1450, disodium phosphate (anhydrous), citric acid, lemon flavor, purified water and oxymetazoline in the concentrations and quantities listed in Table 1 below. Two basic formulations of oxymetazoline were prepared at 0.005% w/v (Basic Formula I) and 0.05% w/v (Basic Formula II). Purified water was added in sufficient quantity to provide a final solution with a volume of 500 milliliters. The pH of the basic formula was measured as pH 4.76.

TABLE 1
Basic Formula I (0.005% w/v) and II (0.05% w/v)

| | Amount (g) per | |
|---|---|---|
| Ingredient | 500 mL batch | % w/v |
| Avicel ® RC 591 (microcrystalline | 15.00 | 3.00 |
| cellulose, carmellose sodium) | | |
| Povidone K29-32 | 45.00 | 3.00 |
| Polyethylene glycol 1450 | 25.00 | 5.00 |
| Disodium phosphate, anhydrous | 1.57 | 0.313 |
| Citric acid | 1.34 | 0.268 |
| Lemon flavor | 1.5 | 0.15 |
| Oxymetazoline HCl | 2.5 mg; or | 0.005; or |
| | 25.0 mg | 0.05 |
| Purified water | quantity sufficient | — |
| | to 500 mL | |

Addition of Transition Metal Additives. To equal volumes of Basic Formula I prepared above, varying quantities of iron sulfate (FeSO₄) were added to assess the effect of different concentrations of iron (II) (Fe²⁺) on oxymetazoline degradation. Six different samples having iron (II) concentrations of 0 ppm, 10 ppm, 25 ppm, 50 ppm, 100 ppm, and 500 ppm added were prepared. The six samples were placed into individual clear glass containers and subjected to controlled light exposure as described below.

ICH Photostability Testing. In order to assess the effect of different transition metal additive concentrations on oxymetazoline degradation, the six samples were subjected to photolytic stress in accordance with ICH Photostability Testing Standards. Samples were exposed to a controlled light source having intensity in the ultraviolet, visible, and infrared spectral regions (ICH Option 1 light source) for a minimum of 1.2 million lux-hours total exposure.

HPLC Analysis. The amounts of oxymetazoline hydrochloride remaining in the final volumes of each sample after photostability testing were determined by gradient HPLC analysis, under the parameters and conditions below:

Injection volume: 25 μL;

Column: Zorbax Eclipse Plus C18, 4.6×150 mm, 3.5 μm;

Column Temperature: 45±2° C.;

UV detection wavelength: 280 nm (4 nm bandwidth)

Mobile Phase-A: 75 mM sodium perchlorate solution, pH=3.0±1;

Mobile Phase-B: methanol;

Linear Gradient Program:

| Time (min) | Flow (mL/min) | % A | % B |
|---|---|---|---|
| 0.0 | 1.0 | 70 | 30 |
| 12.0 | 1.0 | 58 | 42 |
| 12.1 | 1.0 | 30 | 70 |
| 14.0 | 1.0 | 30 | 70 |
| 14.1 | 1.0 | 70 | 30 |
| 19.0 | 1.0 | 70 | 30 |

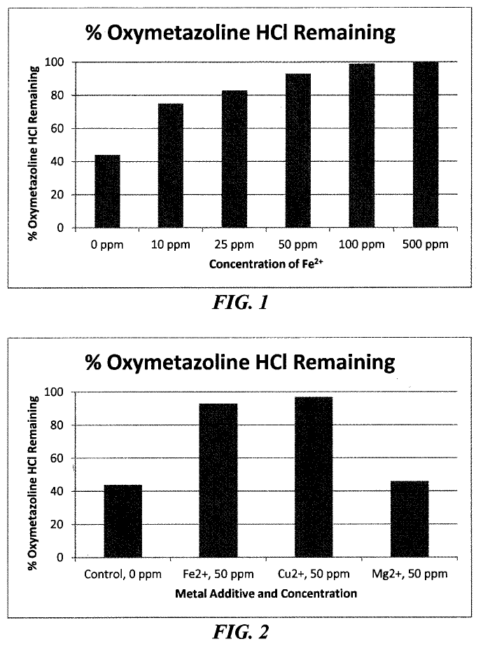

The oxymetazoline concentrations for each sample were calibrated against an HPLC chromatogram of a known sample of oxymetazoline HCl in the basic formulation as a standard, for which concentration was calculated as function of peak integration. The results of the photostability tests for varying concentrations of iron as transition metal additive are shown in Table 2 below and FIG. 1.

TABLE 2

| | | |
|---|---|---|
| Basic Formula I, | Iron (II) Fe²⁺, | % Oxymetazoline HCl |
| Sample No. | concentration | Remaining |

| | | | |
|---|---|---|---|
| 1-1 | 0 | ppm | 44 |
| 1-2 | 10 | ppm | 75 |
| 1-3 | 25 | ppm | 83 |
| 1-4 | 50 | ppm | 93 |
| 1-5 | 100 | ppm | 99 |
| 1-6 | 500 | ppm | 100 |

It was observed that the addition of iron sulfate to the Basic Formula I to provide total transition metal concentrations of at least 10 ppm resulted in a significant reduction in the photo-induced degradation of oxymetazoline. Above concentrations of 50 ppm Fe²⁺, over 90% of the original oxymetazoline concentration was preserved after controlled light exposure.

### Example 2: Variable Transition Metals in Transition Metal Additive on Photolytic Degradation

The effect of different transition metals on the enhanced stability of oxymetazoline was evaluated in Example 2. Basic Formula I was prepared as described in Example 1. To this formulation, iron (II) sulfate (FeSO₄), copper (II) sulfate (CuSO₄), and magnesium chloride (MgCl₂) were added separately to provide three samples each containing a different transition metal additive at a concentration of 50 ppm. The three samples containing different transition metal additives were subjected to controlled light exposure in accordance with ICH Photostability Testing standards in transparent glass containers to determine their effects on the photostability of oxymetazoline hydrochloride. A fourth sample of Basic Formula I without any metal additives was also tested as a control. The light source and total energy exposure conditions employed in Example 2 were identical to the conditions described in Example 1 above.

The amounts of oxymetazoline hydrochloride remaining in the final volumes of each sample after photostability testing were determined by HPLC analysis as in Example 1 above. The results are shown in Table 3 below and FIG. 2.

TABLE 3

| Basic Formula I, | Transition Metal | % Oxymetazoline HCl |
|---|---|---|
| Sample No. | concentration | Remaining |
| 2-1 | Control, 0 ppm | 44 |
| 2-2 | Fe²⁺, 50 ppm | 93 |
| 2-3 | Cu²⁺, 50 ppm | 97 |
| 2-4 | Mg²⁺, 50 ppm | 46 |

The addition of transition metal-based additives iron sulfate and copper sulfate at 50 ppm concentrations were found to achieve similar preservation of oxymetazoline concentrations under controlled light exposure. In contrast, the addition of magnesium chloride to the basic oxymetazoline formulation did not show significant enhancement of oxymetazoline stability against photo-degradation. The quantity of oxymetazoline remaining after photolytic stress in the magnesium-containing sample, that is, less than 50%, was similar to the quantity recorded for the control sample without any metal additive.

### Example 3: Combination Formulations: Transition Metal Additives and Chelating Reagent or Antioxidant Photolytic Degradation

In order to assess any stabilizing effect of chelating agents and antioxidants for photo-induced degradation of oxymetazoline, eleven samples containing combinations of chelating reagents and antioxidants with different concentrations of transition metals were prepared according to Table 4 below, starting from Basic Formula I prepared in Example 1. Iron (II) sulfate was utilized as the transition metal additive for Fe²⁺ samples; iron (III) sulfate was utilized as the transition metal additive for the Fe³⁺ comparative sample. The concentration of EDTA added to the basic formula was 0.1% w/v and the concentration of sodium metabisulfite was 0.006% w/v.

Each sample was subjected to controlled light exposure in accordance with ICH Photostability Testing standards (using the identical light source and total exposure as in Examples 1 and 2 above) in a transparent glass container. The amounts of oxymetazoline hydrochloride remaining in the final volumes of each sample after photostability testing were determined by HPLC analysis as in Example 1 above. The concentration of oxymetazoline HCl degraded was calculated as the difference of the concentration of the basic formulation and the concentration of oxymetazoline remaining in each sample after controlled light exposure.

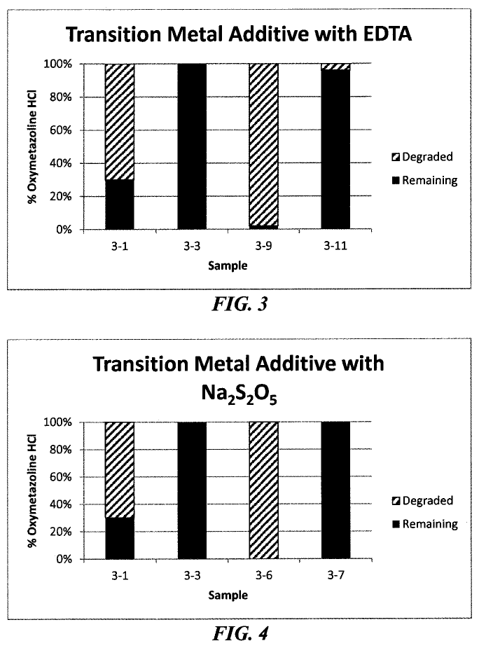

The results are shown in Table 4. FIG. 3 and FIG. 4 show plots of selected results for different combinations of transition metal additives (Fe²⁺, 40 ppm) with EDTA as chelating agent, and with Na₂S₂O₅ as antioxidant, respectively.

TABLE 4

| Basic | EDTA | Na₂S₂O₅ | Added | Added | Added | | |
|---|---|---|---|---|---|---|---|
| Formula I, | added | added | Fe²⁺ | Fe²⁺ | Fe³⁺ | % Oxymetazoline HCl | |
| Sample No. | (0.1%) | (0.006%) | (4 ppm) | (40 ppm) | (40 ppm) | Remaining | Degraded |
| 3-1 | — | — | — | — | — | 30% | 70% |
| 3-2 | — | — | Yes | — | — | 30% | 70% |
| 3-3 | — | — | — | Yes | — | 100% | 0% |
| 3-4 | — | Yes | — | — | — | 20% | 80% |
| 3-5 | — | Yes | — | Yes | — | 100% | 0% |
| 3-6 | — | Yes | — | — | — | 0% | 100% |
| 3-7 | — | Yes | — | Yes | — | 100% | 0% |
| 3-8 | — | Yes | — | — | Yes | 100% | 0% |
| 3-9 | Yes | — | — | — | — | 2% | 98% |
| 3-10 | Yes | — | Yes | — | — | 30% | 70% |
| 3-11 | Yes | — | — | Yes | — | 96% | 4% |

From the different combinations tested, it was observed that added EDTA or added Na₂S₂O₅ alone were not effective in preventing photo-degradation of the oxymetazoline hydrochloride. Similarly, the addition of transition metal additives at a concentration of 4 ppm was insufficient to prevent the majority of oxymetazoline hydrochloride from undergoing photo-induced degradation, and in fact, produced identical results to the basic formulation of oxymetazoline tested alone. It was found, however, that the addition of transition metal additives to provide total transition metal concentrations of 40 ppm of Fe²⁺ effectively prevented the degradation of oxymetazoline and showed near quantitative recovery of the original oxymetazoline concentration. The addition of Fe³⁺ at 40 ppm to the basic oxymetazoline formulation was observed to confer the same photostability enhancement as Fe²⁺ at 40 ppm.

The combined addition of transition metal additives at a concentration of 40 ppm with either a chelating agent (EDTA) or antioxidant (Na₂S₂O₅) was also effective in reducing the degradation of oxymetazoline, with quantitative or near quantitative recovery of the original oxymetazoline concentration.

### Example 4: Packaging Type and Photolytic Degradation Products

To determine the effects of packaging type in attenuating the individual degradation pathways, three samples were prepared according to Table 5 below by adding iron-based transition metal additives (FeSO₄; FeCl₂; and FeCl₂H₂₂O₁₄, iron (II) gluconate) to Basic Formula II of Example 1. A separate sample of the Basic Formula II alone was utilized as a control.

Each of the four samples in Table 5 was placed into a clear glass bottle for photolytic stability testing. The four samples were subjected to controlled light exposure in accordance with ICH Photostability Testing Standards (using the same light source as described in Examples 1-3 above). After photolytic exposure for the requisite period of time, the four samples were analyzed by HPLC to quantify the amount of each degradation product formed. A sample containing known quantities of a oxymetazoline hydrochloride reference standard was used as calibration standard to quantify the concentrations of the degradation products as peak integrations.

TABLE 5

| Sample | | | % OXY | DegA | DegB | DegD |
|---|---|---|---|---|---|---|
| No. | Container | Basic Formula II | Recovery | (%) | (%) | (%) |
| 4-1 | Glass | Control | 95.0 | ND | 0.193 | 2.871 |
| | bottle | | | | | |
| 4-2 | Glass | Added FeSO₄ | 100.2 | ND | 0.044 | ND |
| | bottle | (100 ppm) | | | | |
| 4-3 | Glass | Added FeCl₂ | 101.4 | ND | ND | ND |
| | bottle | (100 ppm) | | | | |
| 4-4 | Glass | Added Fe | 99.1 | ND | ND | ND |
| | bottle | gluconate | | | | |
| | | (100 ppm) | | | | |

No formation of degradation product DegA was observed for any of the samples evaluated under photolytic stress in Table 4. Degradation product DegD was observed to form in the control sample having no transition metal additives. However, the addition of transition metal additives to the Basic Formula II at transition metal concentrations of 100 ppm reduced the quantity of DegD to non-detectable (ND) levels. The formation of DegB was reduced to non-detectable levels in two of the samples containing transition metal additives—iron chloride and iron gluconate. The third sample containing 100 ppm iron sulfate did not eliminate the formation of DegB but significantly reduced the concentration of DegB as compared to the control sample.

The effect of specialized packaging to control light exposure on the degradation of the oxymetazoline formulations of the present disclosure was also examined. The two samples of the Basic Formula II containing FeSO₄ and FeCl₂, respectively, were placed in opaque LDPE bottles to mimic the effect of light-blocking packaging. A control sample of the Basic Formula II was evaluated in a clear glass bottle. The samples in the LDPE bottles and the control were exposed to controlled light in accordance with ICH Photostability Testing standards, using the same light source and exposure conditions as described above. HPLC analyses of the samples and control were conducted to assess the extent of degradation and to identify the degradation products as described above. The results are shown in Table 6 below.

TABLE 6

| Sample | | | % OXY | DegA | DegB | DegD |
|---|---|---|---|---|---|---|
| No. | Container | Basic Formula II | Recovery | (%) | (%) | (%) |
| 5-1 | Glass | Control | 95.0 | ND | 0.193 | 2.871 |
| | bottle | | | | | |
| 5-2 | LDPE | Added FeSO₄ | 101.7 | ND | 0.105 | ND |
| | bottle | (100 ppm) | | | | |
| 5-3 | LDPE | Added FeCl₂ | 101.0 | ND | 0.050 | ND |
| | bottle | (100 ppm) | | | | |

No formation of degradation product DegA was observed under photolytic stress for the control or either sample in the LDPE bottles. Degradation product DegD was observed to form in the control sample, which was placed in a transparent glass bottle and did not contain any transition metal additives. However, the samples containing the transition metal additives, which were further placed in the LDPE bottles, did not show any detectable formation of DegD.

The degradation product DegB was observed to form in the control sample as well as the samples containing the transition metal additives packaged in LDPE bottles. However, the samples containing the transition metal additives and packaged in the LDPE bottles showed significant decreases in the quantity of DegB formed as compared to the control.

### Example 5: Transition Metal Additives and Elevated Temperature Tests

Two samples—a control sample containing the Basic Formula II, and Basic Formula II further containing 100 ppm FeSO₄—were placed in separate glass containers and subjected to an applied heat stress for 14 days. The temperature of the samples was maintained at 75 degrees Celsius throughout the experiment. The samples were evaluated by HPLC analysis at two time different time points during the experiment—after 5 days had elapsed at elevated temperature and after the full course of the experiment (14 days)—for the presence and concentration of oxymetazoline degradation products. The same standard protocol for the HPLC analysis used in Example 4 above to quantify the concentrations of DegA, DegB, and DegD was utilized in the analysis for the samples kept at elevated temperature. The results of the HPLC analysis are shown in Table 7.

TABLE 7

| Sample | Time | | % OXY | DegA | DegB | DegD |
|---|---|---|---|---|---|---|
| No. | (days) | Basic Formula II | Recovery | (%) | (%) | (%) |
| 6-1 | 5 | Control | 101.3 | 0.571 | 0.139 | ND |
| 6-2 | | Added FeSO₄ | 102.9 | 0.461 | ND | ND |
| | | (100 ppm) | | | | |
| 6-3 | 14 | Control | 101.3 | 1.868 | 0.089 | ND |
| 6-4 | | Added FeSO₄ | 100.2 | 1.462 | ND | ND |
| | | (100 ppm) | | | | |

The formation of DegD was not observed under the elevated temperature conditions described here. However, it was observed that the addition of transition metal additives mitigated the formation of degradation product DegA as compared to the control samples and reduced the formation of DegB to non-detectable levels.

What is claimed is:

1. A pharmaceutical formulation, comprising:
0.005% w/v to 0.05% w/v oxymetazoline hydrochloride;
pharmaceutically acceptable excipients; and
one or more transition metal additives,
wherein the pharmaceutical formulation has a total transition metal concentration of at least 10 ppm.

2. The pharmaceutical formulation of claim 1, wherein the total transition metal concentration is between 10 ppm and 500 ppm.

3. The pharmaceutical composition of claim 1, wherein the total transition metal concentration is 25 ppm and 200 ppm.

4. The pharmaceutical formulation of claim 1, at least one of the one or more transition metal additives comprises a transition metal selected from the group consisting of titanium, manganese, iron, cobalt, copper, and zinc.

5. The pharmaceutical formulation of claim 1, wherein at least one of the one or more transition metal additives comprises iron, copper, or zinc.

6. The pharmaceutical formulation of claim 1, at least one of the one or more transition metal additives comprises iron.

7. The pharmaceutical formulation of claim 1, wherein at least one of the one or more transition metal additives comprises a sulfate salt, chloride salt, or a gluconate salt.

8. The pharmaceutical formulation of claim 1, wherein the pharmaceutical formulation further comprises a buffering agent.

9. The pharmaceutical formulation of claim 8, wherein the buffering agent is selected from the group consisting of citric acid, a citrate salt, acetic acid, an acetate salt, phosphoric acid, and a phosphate salt, and any combinations thereof.

10. The pharmaceutical formulation of claim 9, wherein the buffering agent comprises a combination of citric acid and disodium phosphate.

11. The pharmaceutical formulation of claim 1, wherein the pH of the pharmaceutical formulation is between pH 4.00 and pH 5.00.

12. The pharmaceutical formulation of claim 1, wherein the pharmaceutical formulation further comprises a chelating agent.

13. The pharmaceutical formulation of claim 12, wherein the chelating agent comprises an ethylenediaminetetraacetate salt.

14. The pharmaceutical formulation of claim 1, wherein the pharmaceutical formulation further comprises an antioxidant.

15. The pharmaceutical formulation of claim 14, wherein the antioxidant is Na₂S₂O₅.

16. The pharmaceutical formulation of claim 1, wherein the pharmaceutical formulation is aqueous.

17. The pharmaceutical formulation of claim 1, wherein the pharmaceutical formulation has a shelf-life of at least 24 months.

18. A pharmaceutical formulation, comprising:
0.005% w/v to 0.05% oxymetazoline hydrochloride;
pharmaceutically acceptable excipients; and
one or more transition metal additives;
wherein the pharmaceutical formulation has a total transition metal concentration of at least 10 ppm, and
wherein at least 75% oxymetazoline remains after the pharmaceutical formulation is subjected to controlled light exposure in accordance with ICH Photostability Testing standards in a transparent container.

19. The pharmaceutical formulation of claim 18, wherein at least 90% oxymetazoline remains after the pharmaceutical formulation is subjected to controlled light exposure in accordance with ICH Photostability Testing standards.

20. The pharmaceutical formulation of claim 18, wherein the pharmaceutical formulation comprises:
(i) less than 0.1% w/v DegD;
(ii) less than 0.15% w/v DegA; or
(iii) less than 0.1% w/v DegB,
or any combinations thereof, after the pharmaceutical formulation is subjected to controlled light exposure in accordance with ICH Photostability Testing standards.

21. The pharmaceutical formulation of claim 18, wherein the pharmaceutical formulation comprises less than 0.1% w/v DegD after the pharmaceutical formulation is subjected to controlled light exposure in accordance with ICH Photostability Testing standards.

22. The pharmaceutical formulation of claim 18, wherein the pharmaceutical formulation comprises less than 0.15% w/v DegA after the pharmaceutical formulation is subjected to controlled light exposure in accordance with ICH Photostability Testing standards.

23. The pharmaceutical formulation of claim 18, wherein the pharmaceutical formulation comprises less than 0.1% w/v DegB after the pharmaceutical formulation is subjected to controlled light exposure in accordance with ICH Photostability Testing standards.

24. The pharmaceutical formulation of claim 18, wherein the pharmaceutical formulation comprises:
(i) less than 0.1% w/v DegD;
(ii) less than 0.15% w/v DegA; and
(iii) less than 0.1% w/v DegB,
after the pharmaceutical formulation is subjected to controlled light exposure in accordance with ICH Photostability Testing standards.

25. The pharmaceutical formulation of claim 18, wherein the pharmaceutical formulation comprises less than 0.5% w/v total impurities after the pharmaceutical formulation is subjected to controlled light exposure in accordance with ICH Photostability Testing standards.

26. A method of treating sinus congestion, comprising administering to a patient in need of treatment thereof a pharmaceutical formulation according to claim 1.

27. The method of claim 26, wherein the pharmaceutical formulation has a total transition metal concentration of between 25 and 200 ppm.

28. The method of claim 26, wherein the method comprises administering the pharmaceutical formulation via nasal administration.

29. The method of claim 26, wherein the method comprises administering the pharmaceutical formulation as a nasal spray.

30. A nasal spray system, comprising:
a pharmaceutical formulation according to claim 1; and
a container containing the pharmaceutical formulation therein.

31. The nasal spray system of claim 30, wherein the system further comprises:
a pump, wherein the pump is attached to the container and is configured to aerosolize the pharmaceutical formulation; and
a nozzle, wherein the nozzle is attached to the pump and is configured to receive the aerosolized pharmaceutical formulation and deliver the aerosolized pharmaceutical formulation into a nostril or a nasal cavity.

32. The nasal spray system of claim 30, wherein the container is a glass or plastic bottle.

33. The nasal spray system of claim 30, wherein the container is opaque to ultraviolet light.

34. The nasal spray system of claim 30, wherein the container is transparent to ultraviolet light.
